(19) United States
(12) Patent Application Publication
(10) Pub. No.: US 2026/0237485 A1
(43) Pub. Date: Aug. 13, 2026

(54) Baby Care Management Device and Method

(71) Applicant: MEDELA AG, Baar (CH)

(72) Inventors: Angela Groshner, McHenry, IL (US); Johan Thorselius, Solna (SE)

(21) Appl. No.: 19/048,324

(22) Filed: Feb. 7, 2025

(51) Int. Cl. G16H 20/60

(52) U.S. Cl. CPC G16H 20/60

(57) ABSTRACT

The present invention relates to a digital solution supporting the healthcare professional to generate a suitable set of data for the calculation of the composition of the feed for a newborn on the basis of a receipt of the doctor and to select the components of the feed from stock with the best yield of available stock material and minimum waste. The invention proposes a computerized method for preparing a feed for a newborn comprising the steps of inputting via an input interface input information related to the feed for the newborn including an amount of donated milk from a donating lactating mother to be fed to the newborn, energy-related information on the energy of the feed and application-related information on the conditions of applying the feed to the newborn and outputting via an output interface output information on the composition of the feed including components of the feed and the amount of each component of the feed. The invention also proposes a newborn feed preparation device adapted to carry out the inventive method.

### FIELD OF THE INVENTION

The present invention relates to a newborn feed preparation device for preparing a feed for a newborn and a computerized method for preparing a feed for a newborn.

### BACKGROUND OF THE INVENTION

Infants prematurely born are treated in NICU wards. Here infants are fed with feed that allow rapid growth. The feed are made of milk donated from a lactating donor, usually the mother of the newborn, and at least one fortifier and/or infant formula, i.e. a product that increases the calorie content of the milk. Fortification is achieved by adding fortifiers and/or infant formula to the milk, that increase the energy content of the milk or other characteristics of the milk. Traditionally, the doctor prepares a fortification program usually specified in receipts. Such fortification program defines the
amount of donated milk,
fortification (final energy content of the fortified milk i.e. feed (in Kcal/ounce; e.g. 24 Kcal/ounce)
frequency of feeding (e.g. every 3 h)
specific fortifiers and/or infant formula to be used (if any).

### Problems to be Solved

A mistake in the nurse's preparation of the feed could have fatal consequences on the baby, e.g. in case the feed is not properly prepared because e.g. the wrong amount of fortifiers is added to the donated milk. Calculations may be erroneous because the healthcare professional may be multi-tasking or distracted and the receipts can become complex to manually calculate.

### Scope of the Invention

It is an object of the present invention to provide a digital environment that can exclude miscalculation of the components of the feed.

The digital solution can support the healthcare professional to generate a suitable set of data for the calculation of the composition of the feed and optionally to select the components of the feed from stock with the best yield of available stock material and minimum waste.

### DESCRIPTION OF THE INVENTION

At least as a partial solution to the above problems, the present invention provides a newborn feed preparation device and a computerized method for preparing a feed for the newborn.

According to a first aspect of the present invention, there is provided a newborn feed preparation device comprising an input interface configured to receive, from a user, input information related to a receipt of a doctor for feeding a newborn including information on the feed for the newborn and application-related information on the conditions of applying the feed to the newborn, and an output interface configured to output information on the composition of the feed including components of the feed and the amount of each component of the feed. The newborn feed preparation device further comprises a processing circuitry configured by an app to obtain, via the input interface, the input information and to output the output information, which output information is based on the input information and on pre-stored information on available fortifier and/or infant formula and on available donated milk.

The pre-stored information on available fortifier and/or infant formula and on available donated milk usually relates to respective stock at or near the NICU ward. The available fortifier and/or infant formula is usually classified with respect to the amount of fortifier in a fortifier unit. A fortifier unit usually corresponds to a specific amount of the fortifier. This unit is furthermore classified with respect to the type of the fortifier, which information may contain the calorie density of the fortifier of each of the fortifier unit. Such calorie density of each fortifier unit usually belongs to the stored information on the available fortifier. In the present application, available fortifier and/or infant formula is used in singular. However, available fortifier and/or infant formula may not only contain one, but also plural fortifier and/or infant formula units.

The pre-stored information on donated milk usually comprises at least an amount of donated milk per milk unit containing respective donated milk and the type of the donated milk contained in respective milk unit. Respective information on the type may comprises information on the donor of the respective breast milk. Respective breast milk as the base component of the composition to be formulated and mixed for providing the feed may preferably be formed exclusively from milk donated by the mother of the newborn. However, respective feed may likewise comprise milk donated from another lactating person and may even exclusively be formed by milk of such lactating person. Milk of a lactating mother or person usually has a calorie density of 20 Kcal/ounce. Thus, respective calorie density is usually applied when calculating the target calorie density of the feed to be mixed. Where appropriate or required, the information on the type of the donated milk likewise comprises specific information on the energy density of respective donated milk of a specific milk unit. In the present application, available donated milk is used in singular. However, available donated milk may not only contain one, but also plural milk units.

According to a second aspect of the present invention, the same provides a computerized method for preparing a feed for the newborn. The method comprises a step of inputting, via an input interface, input information related to a receipt of a doctor for feeding a newborn including information on the feed for the newborn and application related information on the conditions of the applying the feed to the newborn. The respective computerized method likewise includes a step of outputting, via an output interface, output information on the composition of the feed, including components of the feed and the amount of each component of the feed. Thus, the output information will be suitable to inform the healthcare professional on the components to be used for defining each component of the feed, preferably with respect to each proportion of the component within the feed for finally preparing by mixing the target feed.

When using the computerized method for preparing a feed in accordance with the present invention, the output information is preferably calculated on the basis of standardized information comprising volume of feed, frequency of delivery of the feed, interval time, base composition, base composition calorie density, added fortifier and feed calorie density. Such standardized information may be inputted via the input interface by the doctor formulating the receipt for the newborn. Such a receipt may be formulated on the basis of the standardized information which requires the doctor to formulate the receipt in the specific format of the standardized information.

As mentioned before, the doctor formulating the receipt is rather used to define specific parameters of the feed therapy without taking into account the standardized information facilitating calculation of the output information by means of the computerized method. In view of this, the standardized information is preferably calculated or extracted from the input information, which input information corresponds with the receipt formulated by the doctor in the usual format for the feed therapy. For this, the information put together in the receipt formulated by the doctor may be captured in different ways. Respective information may be captured via a camera of a tablet or smartphone or dedicated device or any other device, which can run the application of the present invention. Respective information can preferably undergo an OCR scan in order to be electrically or digitally readable. The information may be captured via a connection to an EMS, which is an Electronical Medical System of a hospital. The information may likewise be provided via an upload of such EMS. The information may also be manually introduced based on the receipt.

As respective information is rather standardized and includes the amount of available donator milk, fortification of the feed, frequency of feeding, and optionally specific fortifiers which shall be used according to the receipt of the doctor, transferring such input information to calculate the standardized information can reliably be performed in an automated computerized fashion. After capturing respective information, the captured and inputted information may be calculated by an AI system to result in the standardized information, e.g. by AWS (Amazon Web Services) e.g. using the service AWS Bedrock.

The standardized information as an example has the following format:

standardized information

| | | |
|---|---|---|
| volume per feeding | 34 | ounce |
| frequency (one feeding every | 1/3 | 1/h |
| 3 h) | | |
| Interval | 12 | h |
| base composition | mom | |
| | milk | |
| base calorie density | 20 | Kcal/ounce |
| fortifier | Similac | |
| feed calorie density | 24 | Kcal/ounce |

Whereas “Similac” is a brand of infant formula that was developed by Alfred Bosworth of Tufts University and is marketed by Abbott Laboratories. Any other fortifier and/or infant formula may be used and Similac is only provided as an example.

In the standardized information, volume of feed is an amount of volume of feed supplied to the newborn per each feeding. Feeding is an incidence of applying the volume of feed to the newborn, e.g. x ounce with y minutes. Frequency of delivery of feed is the time (e.g. hours) between each feeding occurrence. The interval is the time for supplying the total volume of feed to the newborn. The base composition usually corresponds to the donated milk component of the feed mentioned before. Base calorie density relates to the calorie density of the donated milk, which may either be assumed or measured for each specific unit of the base composition used. Fortifiers are one or more fortifier and/or infant feed contained in the feed. A specific calorie density of the fortifier is usually assigned to each specific fortifier due to the specification/the type of the fortifier. Feed calorie density is the target calorie density of the to be prepared feed.

Based on this standardized information, the output information is preferably calculated by additionally taking into account pre-stored information on available fortifier and available donated milk i.e. base composition. The stored information on available fortifier comprises at least one of the following pieces of information: amount of fortifier per fortifier unit, type of fortifier per fortifier unit and calorie density of the fortifier of respective fortifier unit. The stored information on available donated milk comprises at least one of the following pieces of information:

amount of donated milk per milk unit containing the donated milk and type of donated milk contained in said milk unit. As mentioned before, the calorie density may as well form part of respective stored information for each milk unit. If such information on the calorie density is not provided, the calorie density of the donated milk is assumed to have a calorie density of 20 Kcal/ounce.

The computerized method leads to output information, which identifies at least one elected milk unit among the available donated milk. Respective elected milk unit is calculated as the at least one donated milk unit used to prepare the volume of feed at minimized waste of donated milk. The computerized method may also calculate and/or identify a specific donated milk unit, which meets the requirement of minimum waste of the donated milk used to define the feed and thus mix the feed with donated milk among the stock of donated milk within the entirety of the available donated milk units on stock.

In addition, the computerized method and thus the newborn feed preparation device having a processing circuitry configured to run the computerized method can output output information identifying at least one elected fortifier unit among the available fortifier and/or infant formula. The at least one identified fortifier and/or infant formula is typically identified by the doctor in the receipt. At least one elected fortifier and/or infant formula unit may be calculated as the one or more fortifier and/or infant formula unit used to prepare the volume of feed at minimized waste of the fortifier and/or infant formula. For this to achieve, the computerized method may evaluate any available fortifier and/or infant formula among the stock, which may comprise different units with different amount and/or different calorie density.

According to a preferred embodiment of the present invention, the computerized method for preparing a feed for the newborn includes a calculation step, which calculation step provides output information comprising the following parameters: total volume of feed, total calculated volume of feed, amount of donated milk i.e. base composition in the feed, type of fortifier and/or infant formula, and amount of fortifier and/or infant formula in the feed.

Specifically, the calculation step includes an algorithm that based on the inputs provides as an output the feed to prepare in terms of amount of milk/base composition, optionally at least one fortifier and/or infant formula and the frequency i.e. number of feeds. This calculation step also allows to optimize milk and fortifiers usage, limiting the waste.
Inputs for the calculation step are
the standardized information calculated from the receipt as captured,
optionally the milk unit amounts from the donor mother and possibly other donors (in the example there are available one unit/container with 100 ml human milk, one unit/container with 55 ml and one unit/container with 125 ml milk) from the mom and one unit/container with 75 ml milk donated from other donor,
optionally the available fortifier in terms of quality, specifically calorie density, number of units (e.g. XX unit of Similac® Human Milk Fortifier Extensively Hydrolyzed Protein Concentrated Liquid, each unit having a volume of 5 mLs and a calorie density of 0.8 Kcal/ml that can be mixed each to 25 mLs of 20 kcal/oz milk to create 24 Kcal/oz preparation).

The total volume of feed is usually calculated by multiplying the amount or volume of feed per feeding with the intervals and with the frequency. The output information may be displayed in accordance with the following format:

Output Information

| | | |
|---|---|---|
| Total volume of feed | 136 | ml, from receipt |
| total calculated volume | 150 | ml, calculated |
| volume of base composi- | 125 | ml; elected base composition/milk unit |
| tion/donated milk | | |
| volume of fortifier | 5 | ml; pre-stored information |
| number of units of fortifier | 5 | calculated number, type from pre- |
| | | stored information |

Where
Total volume of feed=volume of feed*interval*frequency=136 ml=34*12/3 ml
150 comes from the instructions of the fortifier (each unit fortifier type XXX having a volume of 5 ml can be mixed to 25 ml milk) and is the amount of selected fortifier suitable to fortify the available donated milk (125 ml; see table below that shows an example for the fortifier Similac quoted above). In the above example, the total calculated volume of 150 ml, volume of fortifier and number of units of fortifier results from a lookup table on ratio based fortifier recipes as follows:

| | 24 Kcalories/fl oz | |
|---|---|---|
| Total Feed | Packets of | ml of human |
| Yield (ml) | fortifier | milk |
| 30 | 1 | 25 |
| 60 | 2 | 50 |
| 90 | 3 | 75 |
| 120 | 4 | 100 |
| 150 | 5 | 125 |
| 180 | 6 | 150 |
| 210 | 7 | 175 |
| 240 | 8 | 200 |
| 270 | 9 | 225 |
| 300 | 10 | 250 |
| 330 | 11 | 275 |
| 360 | 12 | 300 |
| 390 | 13 | 352 |
| 420 | 14 | 350 |
| 450 | 15 | 375 |
| 480 | 16 | 400 |
| 510 | 17 | 425 |
| 540 | 18 | 450 |

The app e.g. calculates all possibilities and keeps as good the one that required the lowest amount of wasted milk. Donated Milk is wasted in the above example because more milk than the required volume is prepared, e.g. above 150-136 ml of milk are wasted.

All possibilities can be calculated with all available milk units on stock.

Application of the invention may work like that:

The doctor formulates a receipt indicating
the minimum volume of feed (e.g. 136 ml)
target calorie density (e.g. 24 Kcalories/ounce)
fortifier(s) and/or infant formula to be used
the app gets a scanned copy (e.g. the software is an app running on a tablet or smartphone) of the receipt and implements the reception step
the standardized information is provided to the calculation step of the software or app the app is also connected to a database with information on donated milk including
default calorie density of mom milk
the app is also connected to a database with information on the fortifiers and/or infant formula, including
default calorie density of different fortifiers
for each fortifier: the amount of feed/the amount of fortifier/the amount of donated milk

The app can run on a mobile device, such as a smartphone or a tablet, or on a PC or laptop. Mobile device may also include a dedicated device.

As derivable from the above description, the present invention provides a computerized method and a device for outputting information on the composition of the to be prepared feed in accordance with the receipt of the doctor. The output information will allow the healthcare professional to prepare the feet in accordance with respective receipt of the doctor at no or drastically reduced risk of preparing a feed not in accordance with the doctors provisions for the feed therapy.

The invention also proposes a newborn feed preparation device adapted to carry out the inventive method. The method steps of the method of the invention may likewise specify the newborn feed preparation device of the invention.

What is claimed is:

1. A newborn feed preparation device for preparing a feed for a newborn comprising:
an input interface configured to receive, from a user, input information related to a receipt of a doctor for feeding a newborn including information on the feed for the newborn including an amount of donated milk from a donating lactating mother to be fed to the newborn, energy related information on the energy of the feed and application-related information on the conditions of applying the feed to the newborn,
an output interface configured to output information on the composition of the feed including components of the feed and the amount of each component of the feed;
processing circuitry configured by an app to:
obtain, via the input interface, the input information; and
based on the input information and on pre-stored information on available fortifier and/or infant formula and on available donated milk, output the output information.

2. A computerized method for preparing a feed for a newborn comprising:
inputting via an input interface input information related to a receipt of a doctor for feeding a newborn including information on the feed for the newborn and application related information on the conditions of the applying the feed to the newborn;
outputting via an output interface output information on the composition of the feed including components of the feed and the amount of each component of the feed.

3. The computerized method for preparing a feed for a newborn of claim 2, wherein the output information is calculated based on standardized information comprising one or more of volume of feed, frequency of delivery of the feed, interval time, donated milk, donated milk calorie density, added fortifier and/or infant formula and feed calorie density.

4. The computerized method for preparing a feed for a newborn of claim 3, wherein the standardized information is calculated from the input information.

5. The computerized method for preparing a feed for a newborn of claim 3, wherein the input information is the standardized information.

6. The computerized method for preparing a feed for a newborn of claim 3, wherein the output information is calculated from the standardized information.

7. The computerized method for preparing a feed for a newborn of claim 6, wherein the standardized information comprises the parameters volume of feed, frequency of delivery of the feed, interval time, base composition, base calorie density, fortifier and/or infant formula, feed calorie density, wherein
volume of feed is the amount or volume of feed supplied to the newborn per feeding,
frequency of delivery of feed is the time between each feeding occurrence,
interval time is time for supplying the total volume of feed to the newborn,
base composition is the donated milk component of the feed,
base calorie density is the calorie density of the donated milk,
fortifier and/or infant formula is one or more fortifier and/or infant formula contained in the base composition,
feed calorie density is the target calorie density of the to be prepared feed.

8. The computerized method for preparing a feed for a newborn of claim 6, wherein the output information is calculated based on pre-stored information on
available fortifier and/or infant formula, wherein the stored information on available fortifier and/or infant formula comprises at least one of: amount of fortifier and/or infant formula per fortifier and/or infant formula unit, type of fortifier and/or infant formula of the fortifier and/r infant formula unit and calorie density of the fortifier and/or infant formula of the fortifier and/or infant formula unit,
and
available donated milk, wherein the stored information on available donated milk comprises at least one of amount of donated milk per milk unit containing the donated milk, type of donated milk contained of said milk unit and calorie density of the donated milk of the milk unit.

9. The computerized method for preparing a feed for a newborn of claim 8, wherein the output information identifies at least one elected milk unit among the available donated milk, wherein the one elected milk unit is calculated as the at least one milk unit used to prepare the total volume of feed having the feed calorie density at minimized waste of donated milk.

10. The computerized method for mixing a feed for a newborn of claim 9, wherein the output information identifies at least one elected fortifier and/or infant formula unit among the available fortifier and/or infant formula, wherein the one elected fortifier and/or infant formula unit is calculated as the least one unit used to prepare the total volume of feed having the feed calorie density at minimized waste of fortifier and/or infant formula.

11. The computerized method for preparing a feed for a newborn of claim 9, wherein the output information comprises total volume of feed, total calculated volume of feed, amount of donated milk in the feed, type of fortifier and/or infant formula and amount of fortifier and/or infant formula in the feed.

12. A computer application (app) adapted to, when run on a mobile device or PC or laptop:
inputting input information related to preparing a feed for a newborn of;
outputting information on the composition of the feed;
processing
input information and
pre-stored information on available fortifier and on available donated milk,
based on the input information and the pre-stored information, outputting the information on the composition of the feed including components of the feed and the amount of each component of the feed.

13. A computer application (app), stored on a computer readable medium, adapted to, when run on a mobile device or PC or laptop, implement the method according to claim 2.
